SEQUENCE LISTING (4 sequences)

SEQ ID NO: 1 - DNA, 1683 bp, Mus musculus
atggatgaagagcgcgccctctacatcgtccgggccggcgaggcgggggctatcgagcgggtcctgagggattacagcgacaagcatagggctactttcaaatttgaatcagcagatgaagataaaagaaagaaactctgtgaaggcatatttaaggtccttgtcaaggaggtcccaacgacatgccaagtgtcctgcctggaagtactccgcattctctccagagacaaaaagatcttagttcctgtaacaactaaggaaaatatgcaaatactgctgagactagccaagctccacgagtcagacgattctttggagaaggtgtcagagttcccagttattgtggaatcattgaagtgtctgtgtaacatcgtgttcaacagtcagatggcacagcagcttagcctggaactgaatctcgctgcaaagctctgtaacctcctaaggaagtgcaaggaccgaaaatttatcaatgacataaagtgctttgacttgcgcttgctcttcgtcttgtcacttttgcacactgacatcaggtcacaattgcgctatgagctccagggactcccgctgctaacccagatcttggaaagtgcctttagcatcaagtggaccgatgagtatgagtcggccatagaccataatggacctcctctctcacctcaggagacagactgtgccattgaggccctcaaagctctgttcaatgtgacggtagacagttggaaggtgcataaagagagcgattctcatcagtttcgtgtcatggcagctgtccttcgccattgtttactaattgtaggtccaactgaagacaaaaccgaagagctgcacagtaatgccgtcaaccttttaagcaatgttccagtctcttgtttggatgttctcatttgtccattaacccatgaagaaacagcccaagaggcagcaactctagatgaactgcccagtgataaaacaacggagaaagacacagctttgaaaaacagtaccatggtatacaatggcatgaatatggaggccattcacgttttactcaattttatggagaagagaattgataagggaagcagctatagagagggtctaactccagttctcagcttattaacagaatgttcccgagcccatcggaacatccgaaaatttctcaaagatcaggttttaccgcctttgagagatgtgacaaatcgaccagaagtcggctcaaccgtgagaaataagctggttcgtctcatgacacatgttgacctcggagtcaagcagattgctgctgagttcctttttgtcctttgcaaagagagagtggatagcctgctgaaatacactggctatgggaacgctgcaggactcttggcggccaggggcctcttggctggaggaagaggagataattggtactcagaggacgaggacacggacactgaagaatacaaaaacgcaaaaccaaacattaatcttatcactggtcatttagaggaaccaatgccaaaccctatagatgaaatgacagaagaacaaaaagaatatgaagccatgaaacttgtcaacatgcttgataaactttccagagaggagttgcttaaaccaatgggactaaaacctgacgggacaataacgcctttggaggaagcactcagccagtattctgtcatcgaagagaccagctcggacacagactga

SEQ ID NO: 2 - DNA, 1146 bp, Mus musculus
atggggtgtttgggcaacagcagcaagaccgcggaagatcagggcgtagatgaaaaagaacgccgcgaggccaacaaaaagatcgagaagcagttgcagaaagagcgcctggcttacaaagcaactcaccgcctgctgcttctgggggctggtgagtccgggaaaagcactatcgtcaaacagatgaggatcctgcacgtcaatggcttcaaccccgaggaaaagaagcagaaaattctggacatcaggaaaaatgtcaaagatgcgatcgtgacaatcgtttcagcaatgagtactatcatacctccagttccactggccaaccctgagaaccagttccggtcagattatatcaagagcatagcccctatcactgactttgaatattcccaggagttctttgaccatgtgaagaagctgtgggacgatgaaggagtgaaggcctgctttgagagatccaacgagtaccagctgatcgactgtgcacaatacttcctggaaaggattgacagtgtcagtctggttgactacacacccacagaccaggacctgctcagatgcagagtgctgacatcaggaatctttgagacacgattccaagtggacaaagtgaactttcacatgtttgatgttggaggccagagagatgagagaagaaaatggatccagtgttttaatgatgtcactgcgatcatttacgtggcggcctgtagtagctacaacatggtgatccgggaagataacaataccaacagacttcgggaatcactggacctgtttgaaagcatctggaataacaggtggttgcgaaccatttctatcatcctattcttgaacaaacaagacatgctggcagaaaaagtcttggcagggaagtcaaaaatcgaagactatttcccggagtatgccaattatactgtccctgaagatgcaacaccagatgcgggagaagatcccaaagttacaagagcaaagttctttatccgggatctgttcttgaggatcagcacagccacgggtgatggcaaacattactgctaccctcacttcacctgcgccgtggacacagagaacatccgcagagtgttcaacgattgccgtgacatcatccagagaatgcatctcaagcagtacgaactcttgtga

SEQ ID NO: 3 - DNA, 792 bp, Mus musculus
atgaggatttttagaccgtggagactgcgctgccctgccttacacttaccctctttccccacgttctctataaagtgtagtttgcctcctcttcccactgacgaagacatgtgtaagagtgtgaccacaggtgagtggaagaaggtcttctacgagaagatggaggaggtgaagccagcggacagctgggacttcatcatagaccccaacctcaagcacaatgtgttggcccctggctggaagcagtacctggaacttcatgcctcaggcaggttccactgttcctggtgctggcacacctggcagtcaccccatgtagtcatcctcttccacatgtacctggacaaggctcagcgcgctggttcggtgcgcatgcgtgtgttcaagcagctctgctacgagtgcggtacagcacggctggatgagtccagcatgctggaggagaacatcgaaagcctggtggacaacctcatcaccagtttgcgagagcagtgctacggggagcgtggtggccactaccgcatccatgtggccagccggcaggacaaccggcgacaccgcggagagttctgcgaggcctgccaggaaggcatcgtgcactggaagcccagtgagaagctgctggaggaggaggcgaccacctacaccttctcccgtgctcccagccccaccaaaccgcaggctgaaacaggctcaggctgcaacttctgctccattccctggtgcttattttgggccacggttttgatgctcatcatctacctgcaattctccttccgtacttctgtctaa

SEQ ID NO: 4 - DNA, 60 bp, Artificial sequence
  coding region for 20 N terminal amino acids of rhodopsin
atgaacgggaccgagggcccaaacttctacgtgcctttctccaacaagacgggcgtggtg

(19) United States
(12) Patent Application Publication
(10) Pub. No.: US 2008/0305547 A1
(43) Pub. Date: Dec. 11, 2008

(54) Method for enhanced functional expression of cell receptors

(76) Inventor: Bettina Malnic, Sao Paulo (BR)

Correspondence Address: FULBRIGHT & JAWORSKI, LLP, 666 FIFTH AVE, NEW YORK, NY, 10103-3198, US

(21) Appl. No.: 11/810,553

(22) Filed: Jun. 6, 2007

(51) Int. Cl. C12N 15/09

(52) U.S. Cl. 435/455

(57) ABSTRACT

The invention relates to methods for enhancing functional expression of receptor molecules in recombinant cells, preferably heterologous cells. In the method, a eukaryotic cell is transformed or transfected with all of the nucleic acid molecule which encodes the receptor, one which encodes a GEF, such as Ric-8A or Ric-8B, and one which encodes Gαolf. The resulting, recombinant cells are then contacted with an agent that stimulates the functional expression of the receptor. Preferably, the receptor is an odorant receptor, or “OR,” and the agent is a ligand for that OR.

### FIELD OF THE INVENTION

The invention relates to methods for improving the expression of molecules in recombinant cells, odorant receptors (“ORs”) in particular. More specifically, it has now been found that Ric-8B, which acts as a guanine nucleotide exchange factor for Gαolf, serves to enhance the expression of ORs in heterologous cells.

### BACKGROUND OF THE INVENTION

Portions of the invention described herein have been disclosed in von Dannecker, et al., Proc. Natl. Acad. Sci. USA, 103(24):9310-9314 (Jun. 13, 2006), and the disclosure of this reference is incorporated by reference in its entirety.

It is well known that mammals can differentiate an untold number of different odorants, with a great degree of sensitivity, and accuracy.

Detection of odorants is accomplished via interaction of odorant molecules with ORs, which are expressed in the cilia of olfactory sensory neurons of the nose. See Buck, et al., Cell, 65:175-187 (1991), incorporated by reference. While the mechanism by which the information received by the ORs is well known, to summarize, this information is transmitted to the olfactory bulb of the brain, and this is then relayed to the olfactory cortex. Mombaerts, et al., Cell 87:675-686 (1996); Ressler, et al., Cell, 79:1245-1255 (1994), and Vassar, et al., Cell, 79:981-991 (1994) show that the information provided by the ORs is organized into a sensory map in the olfactory bulb. The olfactory cortex contains another “map” of OR input, differing from the olfactory bulb “map.” See, Zou, et al., Nature, 414:173-179 (2001).

The ability to discriminate amongst odorants is believed to result from different ligand specificities of ORs. Hence, there is ongoing interest in determining odorant specificities of individual ORs.

The interest, however, and the ability to draw appropriate conclusions about the ORs, has been hampered by an inability to link individual ORs to the odorants they recognize. Malnic, et al., Cell, 96:713-723 (1999); Mombaerts, et al., Nat. Rev. Neurosci., 5:263-278 (2004); Saito, et al., Cell, 119:679-691 (2004), and Shirakova, et al., J. Biol Chem., 280:11807-11815 (2005), collectively incorporated by reference, describe specific ligands for only about 30 ORs. Given that about 1200 OR genes have been identified in mice, and about 388 in humans (Ache, et al., Neuron, 48:417-430 (2005)), this is a significant drawback.

One major reason for this “shortfall,” if not the major reason, is that it has proven to be difficult to obtain functional expression of ORs in heterologous cells. Lu, et al., Traffic, 4:416-433 (2003); McClintock, et al., Neuro. Report, 14:1547-1552 (2003); and Matsunami, et al., Chem. Senses, 30:195-196 (2005) have shown that this is due, in large part, to the fact that ORs cannot reach the plasma membrane.

Previously, Malnic, et al., supra, attempted to address this issue via a combination of Ca²⁺imaging, and single cell RT-PCR, in order to identify ORs expressed by olfactory neurons, when stimulated by different, aliphatic odorants. The approach identified 13 different OR/odorant pairs, and showed that combinatorial receptor codes operate to encode odorant identities.

The skilled artisan will recognize that this approach, while useful, is laborious and time consuming. Given the number of ORs identified to date, the need for more robust systems for this type of work will be evident.

There have been advances which improve expression of ORs in heterologous systems. For example, Kajiya, et al., J. Neurosci., 21:6018-6025 (2002); Krautwurst, et al., Cell, 95:917-926 (1998); and Wetzel, et al., J. Neurosci., 19:7426-7433 (1999), have demonstrated that fusing the 20, N-terminal amino acids of either the rhodopsin or serotonin receptor, to the N-terminal region of ORs, facilitates the surface expression of the ORs. Tagged ORs can then be co-transfected in heterologous cells with Gα15/16, which then promiscuously couples receptors to the phospholipase C pathway. The interaction of odorants with ORs leads to activation, and increased intracellular Ca²⁺concentrations, which are measurable at the single cell level, via use of Ca²⁺sensitive dyes, thus compensating for low OR expression efficiency.

Gaillard, et al., Eur. J. Neurosci., 15:409-418 (2002); Katada, et al., J. Neurochem., 90:1453-1463 (2004); and Spehr, et al., Science, 299:2054-2058 92003), have used this approach with HEK293 cells. Katada, et al., Biochem. Biophys. Res. Comm., 305:964-969 (2003); Levasseur, et al., Eur. J. Biochem., 270:2905-2912 (2003); Matarazzo, et al., Chem. Senses, 30:195-207 (2003); and Shirokova, et al., supra, have demonstrated the efficacy of the method in other cell types.

Shirokova, et al., supra, and Kajiya, et al., supra, have also demonstrated that ORs can couple to endogenous Gαs, or to the olfactory specific molecule Gαolf, in turn leading to odorant induced increases in cAMP, which are monitorable by luciferase reporter gene assays. These assays are very sensitive and allow for high through put analysis of ORs expressed either at low levels, or on a small percentage of cell surfaces.

Shirokova, et al., supra, also noted that coupling of odorant receptors to non-olfactory Gα subunits, like Gα15 and Gα16, can cause altered receptor profiles. One concludes from this that heterologous systems which use endogenous olfactory transduction molecules are more likely to reproduce OR physiological responses.

Saito, et al., supra, have demonstrated that co-expression of ORs with receptor transporting proteins (“RTPs”) hereafter, such as RTP 1 (SEQ ID NO: 3) and 2, and receptor expression enhancing protein (REEP)1 in HEK293T cells promotes OR functional surface expression.

Hague, et al., Proc. Natl. Acad. Sci. USA, 101:13672-13676 (2004), have shown that co-expression of ORs with β2 adrenergic receptor promotes surface expression of the OR known as “M71,” in HEK293 cells. In summary, all of this shows that heterologous expression of ORs can be significantly improved by co-expression with accessory proteins that assist in trafficking of ORs to cell surfaces.

Recently, von Dannecker, et al., J. Neurosci., 25:3793-3800 (2005), incorporated by reference, found that the GEF Ric-8B (SEQ ID NO: 1), interacts with Gαolf. Ric-8B, as a guanine nucleotide exchange factor, or “GEF” catalyzes exchange of GDP for GTP, resulting in an activated form of Gα, which in turn activates a variety of effectors. Von Dannecker, et al., supra, also demonstrated that Ric-8B amplifies dopamine receptor and β2-adrenergic receptor signaling through Gαolf (SEQ ID NO: 2).

It has now been found that Ric-8B promotes functional expression of ORs, in heterologous cells, thus providing a new, robust and efficient system of expression for ORs.

This is a feature of the invention, which will be elaborated upon in grater detail in the disclosure which follows.

### DETAILED DESCRIPTION OF THE PREFERRED EMBODIMENTS

### Example 1

These experiments were carried out in order to determine if Ric-8B amplifies signaling through odorant receptors (“ORs” hereafter). This was accomplished by using one of the murine ORs, i.e., first cloning murine OR-EG (“mOR-EG” hereafter), from murine genomic DNA via polymerase chain reaction (“PCR”), using standard method. Following this, receptor specificity was confirmed by functional expression of the rhodopsin tagged version of mOR-EG, in HEK293T cells. This is explained infra.

HEK293T cells were plated, at 0.5×10⁵ cells/well, of a 96 well plate. The cells were cultured using standard conditions, for 16-20 hours, after which they were transfected, via the well known, lipofectamine method with mOR-EG cDNA that had been subcloned into the XboI and KpnI restriction sites of plasmid pcDNA 3.1 (−), Gαolf whose full length cDNA had been cloned from murine olfactory epithelium, and subcloned into the XboI site of pcDNA 3.1 (−) expression vector, and Ric-8B, whose full length encoding cDNA had been prepared the same way and then cloned into pcDNA3 vector, with a flag epitope, at the BamHI site of pcDNA3 were used. 100 ngs of each cDNA construct were used for transfection. After 3 hours of transfection, serum containing medium was replaced by serum free medium, and the cells were incubated for 40 hours. Then, additional serum free medium containing 300 μM of eugenol was added, after which cells were incubated for 10 minutes. Activity was determined, by using a commercially available, cAMP ELISA kit.

Stimulation of the transfected cells with eugenol increased cAMP levels by approximately a factor of 3, as compared to unstimulated cells, however, those cells which were transfected with untagged mOR-EG and Gαolf did not respond to the agonist at all.

### Example 2

Previously, Saito, et al., Cell, 119:679-694 (2004), incorporated by reference, reported that HEK293T cells, co-transfected with a rhodopsin tagged depicts the rhodopsin tag, i.e., the N-terminal 20 amino acids of rhodopsin (SEQ ID NO: 4) odorant receptor, and RTP1 or RTP2 have exhibited enhanced, odorant dependent cAMP production. Cells which were transfected with untagged mOR-EG, Gαolf, and Ric-8B, showed enhanced cAMP production, in contrast to cells where RTP1 was used instead of Ric-8B. When RTP1 was used as a cotransfectant, full length cDNA was first cloned from murine olfactory epithelium, and then subcloned, into the XhoI and KpnI restriction sites of pcDNA 3.1 (−).

### Example 3

In these experiments, mOR-EG odorant specificity was tested, in cells which had also been transfected with Ric-8B and Gαolf Vanillin, nonenedoic acid, acetophenone, (−), limonene, heptanol, 2-heptanol, hexanol, and (+/−) cerveone were tested at high concentrations (300 μM). Only eugenol stimulated production of cAMP.

The lack of stimulus by vanillin was surprising because vanillin has been shown to stimulate rhodopsin tagged mOR-EG.

The conclusions which can be drawn from these experiments include (i) Ric-8B is able to amplify or signal through Gαolf following activation by a specific ligand. The difference in reactivity with vanillin suggests that tagged mOR-EG has altered structure and hence a specificity differing from the untagged molecule.

### Example 4

Previously, Malnic, et al., Cell, 96:713-723 (1999), incorporated by reference, used single cell, RT-PCR, in order to identify OR sequences from murine olfactory neurons, which had responded to specific aliphatic odorants. One OR, referred to as mOR-S6, responded to nonanedoic acid, but not to the other aliphatic odorants tested. Functional expression of mOR-S6, as well as other mORs, in cells, has proven to be difficult, although Saito, et al., Cell, 119:679-691 (2004), and Shirokaua, et al., J. Biol. Chem., 280:11807-11815 (2005), have reported some success. Given the results in the previous examples, additional experiments were carried out to determine if expression of mOR-S6 could be improved by co-expression with Ric-8B.

The same procedures set forth supra for isolating, cloning, and amplifying mOR-EG were used for the coding region of mOR-S6. Rhodopsin tagged mOR-S6 was also used. The same protocols were carried out as are discussed supra, using nonanedoic acid as the agonist.

The results indicated that, when cells were co-transfected with mOR-S6 and Gαolf, or with these two constructs and Ric-8B, there was no significant increase in cAMP production. When RTP1 replaced Ric-8B, there was a slight, insignificant increase in cAMP production. When all four elements, i.e., the rhodopsin tagged mOR-S6, Ric-8B, RTP1, and Gαolf were used, there was a significant increase in cAMP production; however, this was not observed with non-tagged mOR-S6, used in parallel expression systems. The results also show that Ric-8B acts synergistically with rho tags and RTP, to promote functional expression of ORs that are more difficult to express.

### Example 5

A further set of experiments were carried out, using odorant receptor mOR-17. Previously, Zhao, et al., Science, 279:237-242 (1998), have shown that rat OR-17 recognizes octanol; however, Krautwurst, et al., Cell, 95:917-926 (1998), have shown that the murine form of OR-17 preferentially recognizes heptanal, in a co-expression system with Gα/16, in HEK293 cells. Bozza, et al., J. Neurosci, 22:3033-3043 (2002), showed that, when GFP tagged, mouse olfactory neurons which endogenously express OR-17, the receptor recognized heptanal.

To further this work, the same protocols as are described supra were carried out, using untagged mOR-17, Gαolf, Ric-8B, and RTP1.

When mOR-17, Gαolf, and Ric-8B were used, the transfected cells responded to heptanal; however, when RTP1 replaced Ric-8B, they did not. When all four elements were used, the cells responded to heptanal specifically as well.

These results show that Ric-8B is very useful in augmenting functional expression of odorant receptors, when they are not tagged with a molecule like the rhodopsin tag, confirming what was seen in Example 2, supra, with mOR-E6.

### Example 6

A final set of experiments were designed to determine if Ric-8B impacts cellular localization of Gαolf in the transfected cells. von Dannecker, et al., J. Neurosci., 25:3793-3800 (2005), showed that Ric-8B interacts directly with Gαolf. Cells were plated on chamber slides, and were then transfected, with Gαolf alone, or Gαolf and Ric-8B, using the lipofectamine method discussed supra. They were then fixed, in 3.7% paraformaldehyde in PBS, for 15 minutes, at room temperature. Fixed cells were permeabelized with 0.01% Triton x-100, in a blocking buffer of 5% normal horse serum, and 2% BSA, in PBS, for 1 hour, at room temperature. The cells were incubated, in 2-fold diluted blocking buffer containing 1:500 anti-FLAG, or 1:80 anti-Gαolf antibodies. Fluorescent, secondary antibodies were then used to detect either FLAG or Gαolf. Cells were also counterstained, with dye, to visualize nuclei.

These experiments showed that co-expression of Gαolf with Ric-8B increased the number of cells which showed strong, peripheral localization of Gαolf, as compared to expression of Gαolf alone.

Parallel experiments were carried out where Ric-8BΔ9 was used, which is known to not interact with Gαolf. See von Dannecker, et al., supra. There was no increase in the number of cells showing strong peripheral staining.

The foregoing examples set forth particulars of the invention, which relates to a method for expressing functional receptors, such as odorant receptors, in eukaryotic cells, by co-transforming or co-transfecting the cells with nucleic acid molecules which encode (i) the receptor, (ii) Gαolf, and (iii) a GEF, such as Ric-8B or Ric-8A, followed by contact with a ligand specific for the receptor. The receptor is preferably an odorant receptor, which the coding molecule for which is untagged, “tagged” with, e.g., a portion of rhodopsin or serotonin coding sequence, or some other tag that facilitates expression of a functional receptor.

The method thus lends itself to expressing receptors whose ligand is known, such as those indicated herein, as well as the myriad of receptors whose ligand has not been determined, thereby enabling the ability to determine their partner ligand. In the case of those ORs for which a ligand is already known, the methods of the invention permits, e.g., screening for molecules which either enhance or inhibit functional receptor expression, which is useful in fields such as in agriculture, the food industry, etc.

Other aspects of the invention will be clear to the skilled artisan and need not be reiterated here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

We claim:

1. A method for functional expression of a receptor molecule in a recombinant cell, comprising transforming or transfecting a eukaryotic cell with (i) a nucleic acid molecule which encodes a receptor, (ii) a nucleic acid molecule which encodes a GEF, and (iii) a nucleic acid molecule which encodes Gαolf, and contacting said eukaryotic cell with an agent which stimulates functional expression of said receptor.

2. The method of claim 1, wherein said GEF is Ric-8A or Ric-8B.

3. The method of claim 2, wherein said GEF is Ric-8B.

4. The method of claim 1, wherein said receptor is an odorant receptor.

5. The method of claim 1, wherein (i) is tagged with a nucleic acid molecule which enhances its expression.

6. The method of claim 1, further comprising transforming or transfecting said eukaryotic cell with RTP-1.

7. The method of claim 5, wherein (i) is tagged with a portion of a nucleic acid molecule which encodes rhodopsin or serotonin.

8. The method of claim 1, wherein said eukaryotic cell is a mammalian cell.

9. The method of claim 1, wherein said cell is an HEK293 cell or an X.laevis cell.

10. The method of claim 4, wherein said odorant receptor is a human odorant receptor.

11. The method of claim 4, wherein said odorant receptor is an insect odorant receptor.
